(19) United States
(12) Patent Application Publication
(10) Pub. No.: US 2015/0291581 A1
(43) Pub. Date: Oct. 15, 2015

(54) AMORPHOUS FORMS OF PALONOSETRON HYDROCHLORIDE

(71) Applicant: HELSINN HEALTHCARE SA, Lugano/Pazzallo (CH)

(72) Inventors: Waldo MOSSI, Mendrisio (TI) (CH); Giorgio CALDERARI, Rancate (CH); Wilma TAMRAZ, San Jose, CA (US); Tai Wah CHAN, Palo Alto, CA (US)

(73) Assignee: HELSINN HEALTHCARE SA, Lugano/Pazzallo (CH)

(21) Appl. No.: 14/686,887

(22) Filed: Apr. 15, 2015

Related U.S. Application Data
(63) Continuation of application No. 14/044,661, filed on Oct. 2, 2013, now abandoned.
Application No. 14/044,661 is a continuation of application No. 12/513,589, filed on Jan. 7, 2010, now abandoned.
(60) Provisional application No. 60/873,656, filed on Dec. 7, 2006.

(51) Int. Cl. C07D 453/02

(52) U.S. Cl. CPC C07D 453/02

(57) ABSTRACT

Amorphous and polymorphic of palonosetron hydrochloride are disclosed that can be characterized by X-ray powder diffraction patterns, thermal properties, purity and methods of manufacture. These forms of palonosetron hydrochloride can be produced from solution or by solid state interconversions. The forms can be used in pharmaceutical formulations: particularly preferred uses of these formulations are in prevention and treatment of nausea and emesis arising from chemotherapy or postoperative side effects. The forms can optionally be used as mixtures of the crystalline and/or amorphous forms.

### FIELD OF THE INVENTION

The present invention relates to crystalline and amorphous forms of palonosetron hydrochloride, mixtures thereof, pharmaceutical compositions thereof, and uses for such forms and compositions.

### BACKGROUND OF THE INVENTION

The nausea and emetogenic side effects of anti-cancer chemotherapy and radiotherapy are a widespread and longstanding problem. Perhaps less well known but no less important are post-operative nausea and emesis, which may have physiological mechanisms related to the effects seen for chemotherapy. Palonosetron hydrochloride has recently emerged as a highly efficacious anti-nauseant and anti-emetic for use with emetogenic anti-cancer chemotherapies. (Macciocchi, A., et al., “A Phase II dose-ranging study to assesses single intravenous doses of palonosetron for the prevention of highly emetogenic chemotherapy-induced nausea and vomiting,” Proc. Am. Soc. Clin. Oncol., 2002; Abstract 1480. Palonosetron also prevents postoperative nausea and vomiting. (Chelly, J., et al., “Oral RS-25259 prevents postoperative nausea and vomiting following laparoscopic surgery,” Anesthesiol., 85(Suppl. 21):abstract no. 2A (1996)).

Palonosetron is selective, showing a high affinity as an antagonist for the 5-hydroxyltryptamine 3 receptor precursor (5-HT₃ receptor), and showing a low affinity for other receptors such as dopamine receptors (Wong, E. H. F., et al., “The interaction of RS 25259-197, a potent and selective antagonist, with 5-HT₃ receptors, in vitro,” Br. J. Pharmacol., 114:851-859 (1995); Eglen, R. M., et al., “Pharmacological characterization of RS 25259-197, a potent and selective antagonist, with 5-HT₃ receptors, in vivo,” Br. J. Pharmacol., 114:860-866 (1995)). Palonosetron is a synthetic compound existing as a single isomer, and is administered as the hydrochloride salt, as represented in the following structure:

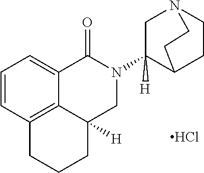 [SMILES: O=C1c2cccc3c2[C@H](CCC3)CN1[C@@H]1CN2CCC1CC2 ; Cl]

The official chemical name for the drug is (3aS)-2-[(S)-1-Azabicyclo[2.2.2]oct-3-yl]-2,3,3a,4,5,6-hexahydro-1-oxo-1Hbenz[de]isoquinoline hydrochloride (CAS No. 119904-90-4); its empirical formula is C₁₉H₂₄N₂O.HCl, and its molecular weight is 332.87. The compound is a white to off-white crystalline powder with a reported melting or decomposition temperature of about 303° C.; it is freely soluble in water, soluble in propylene glycol, and slightly soluble in ethanol and 2-propanol; it has been crystallized by cooling and chilling hot solutions of the latter two solvents, as reported in U.S. Pat. Nos. 5,510,486 and 5,567,818.

A stereoismer of palonosetron hydrochloride also exists, often as an impurity from the manufacturing process for palonosetron hydrochloride, having the following chemical structure:

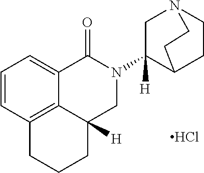 [SMILES: O=C1c2cccc3c2[C@@H](CCC3)CN1[C@@H]1CN2CCC1CC2 ; Cl]

The chemical name of this stereoisomer is (3aR)-2-[(S)-1-Azabicyclo[2.2.2]oct-3-yl]-2,3,3a,4,5,6-hexahydro-1-oxo-1Hbenz[de]isoquinoline hydrochloride, and for purposes of this application the compound will be referred to as the diastereomer of palonosetron hydrochloride.

The unreduced synthetic precursor to palonosetron hydrochloride, which is also often found as an impurity from the manufacturing process of palonosetron hydrochloride, is 2-[(3S)-1-Azabicyclo[2.2.2]oct-3-yl]-2,4,5,6-tetrahydro-1H-benzo[de]isoquinoline-1-one hydrochloride, having the following chemical structure:

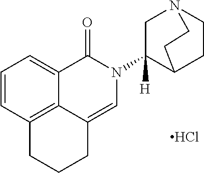 [SMILES: O=c1c2cccc3c2c(cn1[C@@H]1CN2CCC1CC2)CCC3 ; Cl]

The synthesis of palonosetron was reported by B. A. Kowalczyk and C. A. Dvorak in “Total synthesis of the 5-HT₃ receptor antagonist Palonosetron,” Synthesis, 7:816-818 (1996). Kowalczyk and coworkers have taught additional details of the properties, synthesis and diastereomeric separation of palonosetron and related compounds in U.S. Pat. Nos. 5,202,333; 5,510,486; 5,567,818; and 5,576,434.

The ability of a compound to exist in different crystal structures is known as polymorphism. While polymorphs have the same chemical composition, they differ in packing and geometrical arrangement, and exhibit different physical properties such as melting point, shape, color, density, hardness, deformability, stability, dissolution, and the like. Depending on their temperature-stability relationship, two polymorphs may be either monotropic or enantiotropic. For a monotropic system, the relative stability between the two solid phases remains unchanged as the temperature is changed. In contrast, in an enantiotropic system there exists a transition temperature at which the stability of the two phases reverse. (Theory and Origin of Polymorphism in “Polymorphism in Pharmaceutical Solids” (1999) ISBN:)-8247-0237).

Additional polymorphs and other crystalline forms of palonosetron hydrochloride could have commercial value in manufacturing or other applications. It is therefore an objective of this invention to provide novel polymorphic and other forms of palonosetron hydrochloride.

It is another objective to provide novel methods for the preparation and isolation of polymorphic and other forms of palonosetron hydrochloride.

It is still another objective of the invention to provide therapeutic uses of palonosetron hydrochloride polymorphs and other forms of palonosetron hydrochloride.

It is an additional objective to provide pharmaceutical formulations of palonosetron hydrochloride polymorphs and other forms of palonosetron hydrochloride.

### SUMMARY OF THE INVENTION

An amorphous form and two crystalline forms of palonosetron hydrochloride (PH) are provided that can be distinguished from one another by X-ray powder diffraction patterns, thermal properties, purity, and methods of manufacture. These morphological forms of PH can be interconverted and can be used in numerous pharmaceutical compositions.

Therefore, in one embodiment the invention provides an isolated composition of palonosetron hydrochloride comprising Form I PH, Form II PH, amorphous PH, or a mixture thereof, having a melting point at atmospheric pressure of greater than 303° C.

In another embodiment the invention provides an isolated composition of palonosetron hydrochloride comprising Form I PH, Form II PH, amorphous PH, or a mixture thereof, comprising less than or equal to 0.5 wt. % diastereomer.

In yet another embodiment, the invention provides an isolated composition of palonosetron hydrochloride comprising Form I PH, Form II PH, amorphous PH, or a mixture thereof, and less than or equal to 1 wt. % diastereomer, wherein said composition has a melting point of less than 303° C.

In a further embodiment, the invention provides novel methods of making the foregoing PH crystalline forms and mixtures of crystalline forms.

In yet another embodiment, the invention provides pharmaceutical compositions comprising or made from any one of the foregoing PH crystalline forms or mixtures of crystalline forms.

In still further embodiments, the invention provides methods of using any one of the foregoing PH crystalline forms or mixtures of crystalline forms in the treatment of emesis, including PONV, CINV and RINV.

Additional embodiments and advantages of the invention will be set forth in part in the description which follows, and in part will be obvious from the description, or may be learned by practice of the invention. The embodiments and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE FIGURES

The accompanying drawings, which are incorporated in and constitute a part of this specification, illustrate several embodiments of the invention and together with the description, serve to explain the principles of the invention.

FIG. 1A is a graphical depiction of a DSC (“differential scanning calorimetry”) scan of palonosetron hydrochloride as pure Form I, with an endotherm of 313° C.

FIG. 1B is a graphical depiction of a DSC scan of palonosetron hydrochloride as pure Form II, with an endotherm of 311.7° C.

FIG. 1C is a graphical depiction of a DSC scan of pure amorphous palonosetron hydrochloride, with a glass transition endotherm at ca. 40° C., crystallization exotherm commencing at ca. 155° C., first exothermic maximum at ca. 163° C., lesser exothermic maximum at ca. 172° C. exotherm peak, crystallization essentially complete at ca. 178° C., and crystalline melting endotherm with a maximum exceeding 310° C.

FIG. 2A is a graphical depiction of a powder XRDP (“X-ray diffraction pattern”) for palonosetron hydrochloride as pure crystalline Form I, with characteristic maxima at angular positions (two theta): 10.38°±0.1°, 12.04°±0.1°, 14.40°±0.1°, 15.74°±0.1°, 16.89°±0.1°, 17.16°±0.1°, 19.62°±0.1°, 20.88°±0.1°, 23.70°±0.1°, 24.02°±0.1°, 24.73°±0.1° and 25.31°±0.1°.

FIG. 2B is a graphical depiction of a powder XRDP for palonosetron hydrochloride as pure crystalline Form II, with characteristic maxima at angular positions (two theta): 9.92°±0.1°, 1135°±0.1°, 12.98°±0.1°, 15.38°±0.1°, 16.14°±0.1°, 17.51°±0.1°, 25.08°±0.1°, 28.89°±0.1°, 29.66°±0.1° and 32.39°±0.1°.

FIG. 2C is a graphical depiction of a powder XRDP for palonosetron hydrochloride as pure amorphous material, with characteristic maxima (two theta) as diffuse, low-intensity peaks at angular positions (two theta): 16°±0.3° and 21°±0.3°.

### DETAILED DESCRIPTION OF THE INVENTION

The present invention may be understood more readily by reference to the following detailed description of preferred embodiments of the invention and the Examples included therein.

### Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “an active pharmaceutical agent” includes more than one active pharmaceutical agent, and the like.

“Treating” or “treatment” of a disease includes (1) preventing the disease from occurring in an animal that may be predisposed to the disease but does not yet experience or display symptoms of the disease, (2) inhibiting the disease, i.e. arresting its development, or (3) relieving the disease, i.e. causing regression of the disease.

As used herein, the term “crystalline purity,” when used in reference to a crystalline form of palonosetron hydrochloride, refers to the percentage of the crystalline form relative to another crystalline form or an amorphous form of palonosetron hydrochloride in the referenced composition. Thus, for example, a composition comprising Form I palonosetron hydrochloride having a crystalline purity of 95% would comprise 95 weight parts Form I palonosetron hydrochloride and 5 weight parts of other crystalline/amorphous forms of PH.

As used herein, the term “isolated” refers to a chemical state well known among pharmaceutical chemists wherein the recited pharmaceutical ingredient has been separated from the medium in which it was created into a relatively pure state (typically greater than 95 wt. %, 97 wt. % or 98 wt. % pure), before it is mixed with other pharmaceutical ingredients.

“Ampoule” means a small sealed container of medication that is used one time only, and includes breakable and non-breakable glass ampoules, breakable plastic ampoules, miniature screw-top jars, and any other type of container of a size capable of holding only one unit dose of palonosetron (typically about 5 mls.).

“Emesis”, for the purposes of this application, will have a meaning that is broader than the normal, dictionary definition and includes not only vomiting, but also nausea and retching.

“Moderately emetogenic chemotherapy” refers to chemotherapy in which the emetogenic potential is comparable or equivalent to the emetogenic potential of carboplatin, cisplatin ≦50 mg/m², cyclophosphamide <1500 mg/m², doxorubicin >25 mg/ms, epirubicin, irinotecan, or methotrexate >250 mg/m².

“Highly emetogenic chemotherapy” refers to chemotherapy in which the emetogenic potential is comparable or equivalent to the emetogenic potential of cisplatin ≧60 mg/m², cyclophosphamide >1500 mg/m², or dacarbazine.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Therapeutically effective amount” means that amount which, when administered to an animal for treating a disease, is sufficient to effect such treatment for the disease.

### Discussion

Two new crystalline polymorphic forms and an amorphous form of palonosetron hydrochloride (PH) are provided that can be distinguished from other forms or phases of PH by X-ray diffraction patterns, thermal properties, purity, and the methods by which they are made. These crystalline and amorphous forms of PH can be used as intermediates in the manufacture of PH, or can be formulated into pharmaceutical compositions and used for the prevention and treatment of nausea and emesis. The new crystalline polymorphic forms and amorphous form of PH are readily formed and interconvertible by control of the temperature and other laboratory conditions.

In a first embodiment, therefore, the invention relates to purified PH crystals of the present invention, and provides an isolated composition of palonosetron hydrochloride comprising Form I PH, Form II PH, amorphous PH, or a mixture thereof, having a melting point at atmospheric pressure of greater than 303° C. The palonosetron hydrochloride may be Form I, Form II, or amorphous, all as described in greater detail herein. In a second embodiment, the invention relates to purified PH crystals of the present invention, and provides an isolated composition of palonosetron hydrochloride comprising Form I PH, Form II PH, amorphous PH, or a mixture thereof, comprising less than or equal to 0.5 wt. % diastereomer of palonosetron hydrochloride. In yet another embodiment the invention provides an isolated composition of palonosetron hydrochloride comprising Form I PH, Form II PH, amorphous PH, or a mixture thereof, and less than or equal to 1.0 wt. % diastereomer, wherein said composition has a melting point of less than 303° C.

In any of the foregoing embodiments, the composition is preferably in the form of a powder that can be constituted into a finished dosage form, and preferably comprises less than or equal to about 5.0, 3.0, 2.0 or even 1.0% by weight of impurities and degradation products. In an alternative embodiment, which again applies to any of the foregoing embodiments, the composition preferably comprises less than or equal to about 1.0 or 0.5 by weight diastereomer of PH, unreduced synthetic precursor of PH, or a combination thereof (both as described above). A particularly preferred method of making palonosetron hydrochloride having a high degree of purity is to recrystallize palonosetron hydrochloride from a solution of palonosetron hydrochloride in a lower alcohol (i.e. C₁₋₄). In a preferred embodiment, the solution is ethanol or isopropanol. In a particularly preferred embodiment, the alcohol is practically or entirely free of water (i.e. containing no water or no more than about 2.0, 1.0, 0.5 0.1, or 0.05 wt. % water).

In any of the foregoing embodiments, the composition may further be characterized by a substantial degree of crystalline purity. Thus, for example, the composition may comprise Form I PH, Form II PH, or amorphous PH having a crystalline purity of greater than 60%, 70%, 80%, 85%, 90%, 95%, 98%, or even 99%. In like manner, the foregoing compositions may be characterized by melting temperature, which in alternative embodiments is greater than 303° C., 305° C., 308° C., 310° C., or 312° C.

In another embodiment, the invention relates to processes for making finished dosage forms from the PH compositions of the present invention, and to finished dosage forms made by the processes. An exemplary process comprises admixing a composition of palonosetron hydrochloride and a pharmaceutically acceptable carrier, and can be used to make practically any of the dosage forms described elsewhere in this document. In a preferred embodiment, the method of making the finished dosage forms of the present invention further comprises compounding said pharmaceutical formulation into one or more pharmaceutical dosage forms. A preferred dosage form is a sterile injectable liquid, and the preferred pharmaceutically acceptable carrier is water. Another preferred dosage form is a capsule or a liquid-filled capsule, wherein a preferred pharmaceutically acceptable carrier is again water.

### Form I Palonosetron Hydrochloride

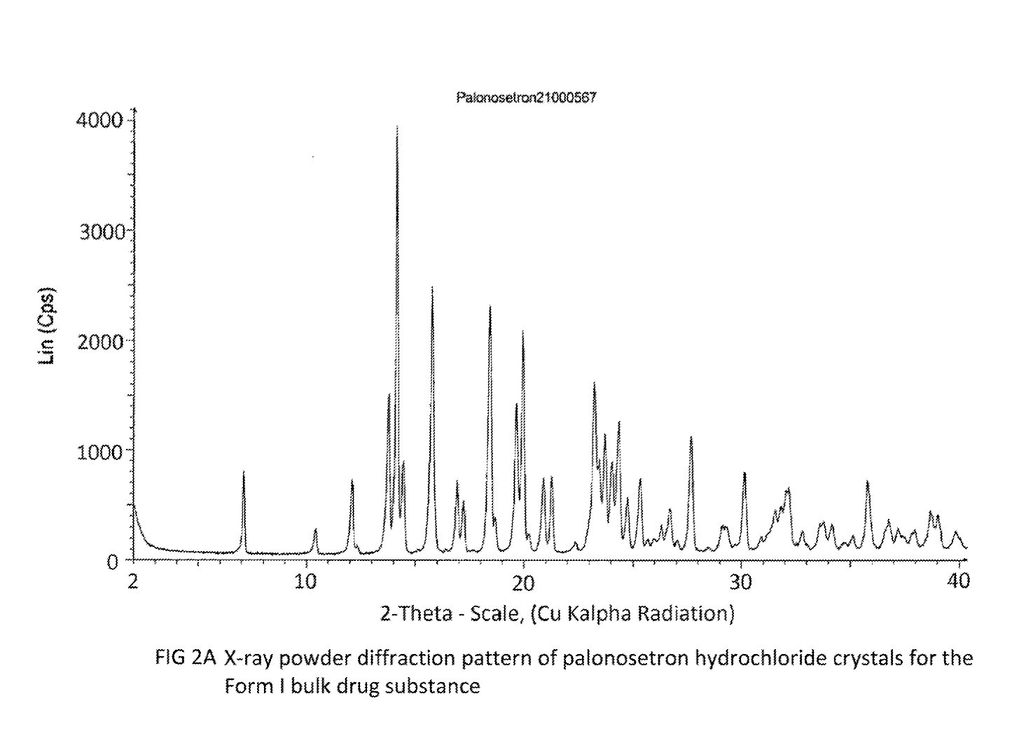

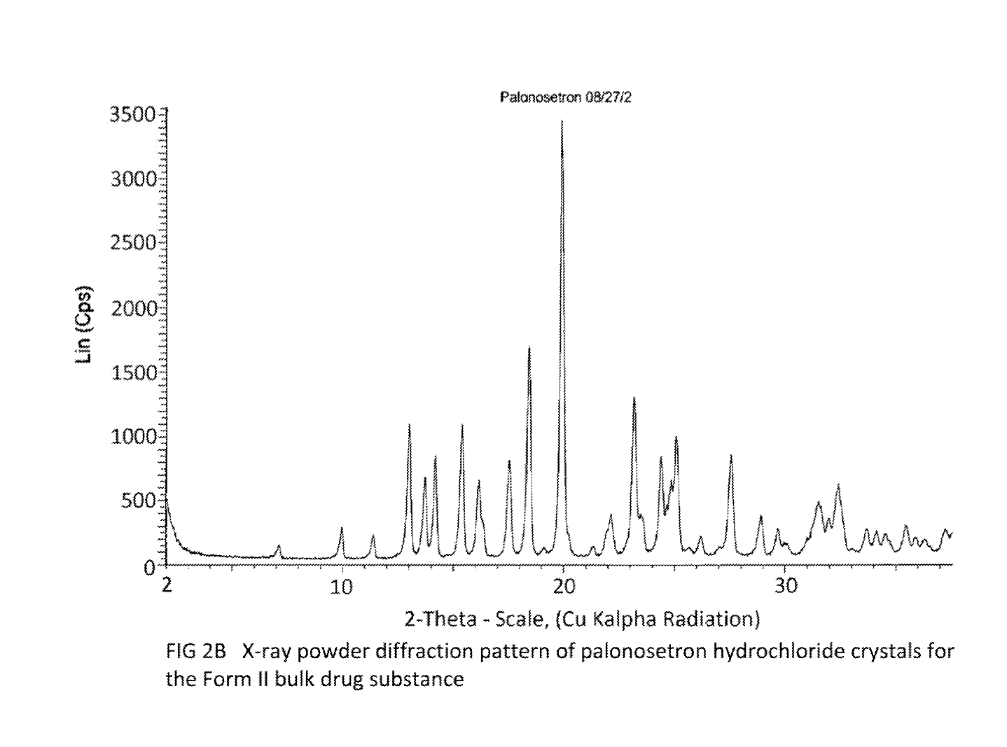

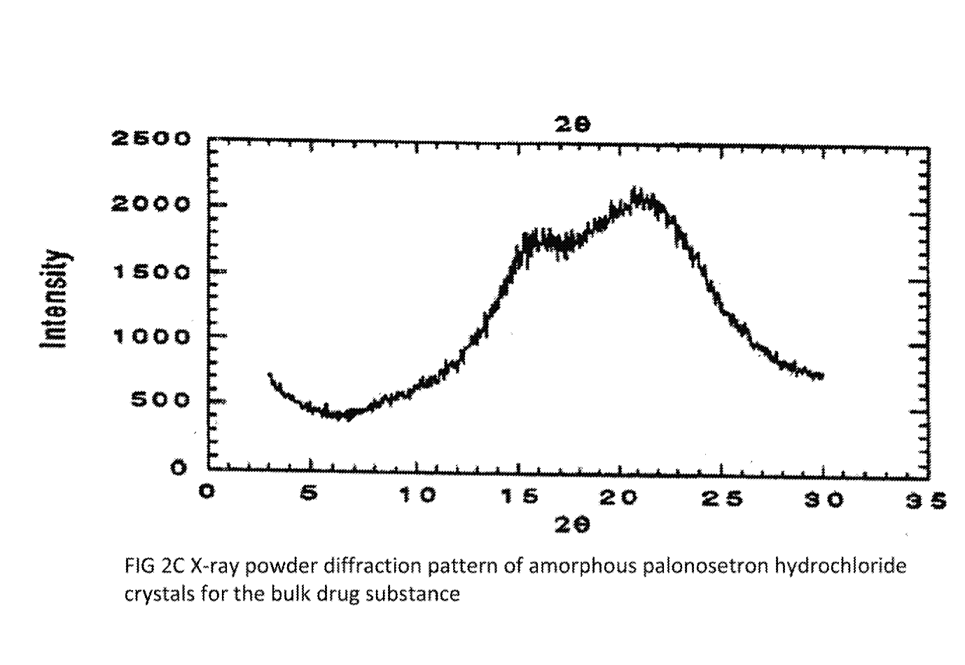

Like all polymorphs, Form I PH crystals can be characterized by the powder diffraction pattern they exhibit when subjected to powder X-ray crystallography, as shown in FIG. 2A. Angular positions (two theta) of characteristic peaks in the powder X-ray diffraction pattern of Form I PH, shown in FIG. 2A, are: 10.38°±0.1°, 12.04°±0.1°, 14.40°±0.1°, 15.74°±0.1°, 16.89°±0.1°, 17.16°±0.1°, 19.62°±0.1°, 20.88°±0.1°, 23.70°±0.1°, 24.02°±0.1°, 24.73°±0.1° and 25.31°±0.1°. Of course, it will be understood that any one or combination of the foregoing peaks can be used to characterize Form I specifically, because each of the peaks distinguishes Form I from Form II. It will also be understood that any one or combination of peaks given in Table 1 can be used to characterize Form I when peak intensity is taken into consideration. Preferred characteristic peaks include 10.38°±0.1°, 12.04°±0.1°, and 15.74°±0.1°, and combinations thereof.

Form I PH crystals can also be characterized by their melting temperature and/or heat of fusion. Thus, Form I PH can also be characterized as a crystalline form of PH having a melting temperature of about 313° C. at atmospheric pressure, when tested according to the methods described herein.

Form I PH crystals can also be characterized by the method(s) used to obtain them. Thus, for example, the Form I PH may be defined as PH crystals obtained by suspending Form II PH crystals, or a mixture of Form I and Form II crystals, in aqueous ethanol or isopropanol at a temperature (preferably about 25° C.) and for a time (typically from one to seven days) sufficient to convert the Form H crystals to Form I. Without being bound by theory, it is believed that this transition occurs either by a gradual dissolution and recrystallization process, or by a solid state reformation facilitated by transient crystal formation containing guest solvent molecules.

The Form I PH may also be defined as PH crystals obtained by exposing the amorphous form of PH to low heat and high relative humidity for several days. Methods for obtaining Form II PH crystals are described herein; methods for obtaining mixtures of Form I and Form II crystals are described, for example, in U.S. Pat. No. 5,510,486.

### Form II Palonosetron Hydrochloride

Form II PH is another polymorph of PH, and can also be characterized by the powder diffraction pattern it exhibits when subjected to powder X-ray crystallography, as shown in FIG. 2B. The angular positions (two theta) of the characteristic peaks in the powder X-ray diffraction pattern of Form II PH, shown in FIG. 2B, are: 9.92°±0.1°, 11.35°±0.1°, 12.98°±0.1°, 15.38°±0.1°, 16.14°±0.1°, 17.51°±0.1°, 25.08°±0.1°, 28.89°±0.1°, 29.66°±0.1° and 32.39°±0.1°. Of course, it will be understood that any one or combination of the foregoing peaks can be used to characterize Form I specifically, because each of the peaks distinguishes Form II from Form I. It will also be understood that any of the peaks given in Table 3 can be used to characterize Form II when peak intensities are taken into consideration. Preferred characteristic peaks include 9.92°±0.1°, 12.98°±0.1°, 15.38°±0.1°, 16.14°±0.1° and 17.51°±0.1°, and combinations thereof.

Form II PH crystals can also be characterized by their melting temperature and/or heat of fusion. Thus, Form II PH can also be characterized as a crystalline form of PH having a melting temperature of about 311.7° C. at atmospheric pressure, when tested according to the methods described herein.

Form II PH crystals can also be characterized by the method(s) for making them. Thus, for example, Form II PH crystals can be defined as the PH crystals obtained by crystallization from a hot (i.e. greater than 40, 50 or 60° C.) low molecular weight alcoholic (i.e. C₁₋₄, preferably ethanolic) solution of dissolved PH. Alternatively, the Form II PH crystals can be defined as the crystalline form of PH obtained when palonosetron base is precipitated with hydrochloric acid from a low molecular weight alcoholic (i.e. C₁₋₄, preferably ethanolic) solution.

### Amorphous Palonosetron Hydrochloride

Amorphous PH is another form of PH which, by definition, is non-crystalline. As shown in FIG. 2C, the form is characterized by the lack of any true characteristic peaks when analyzed by x-ray diffraction, although diffuse, low intensity peaks occur at angular positions (two theta) of 16°±0.3°, and 21°±0.3°.

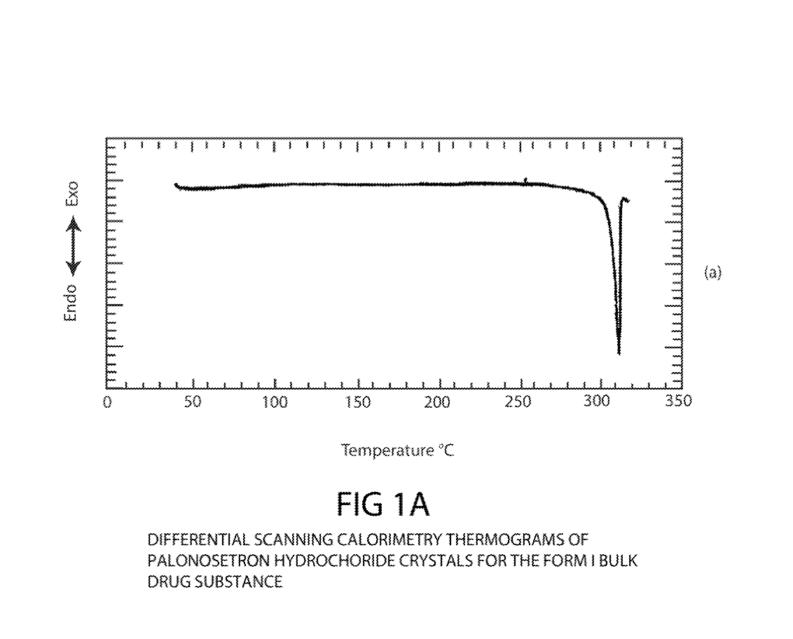

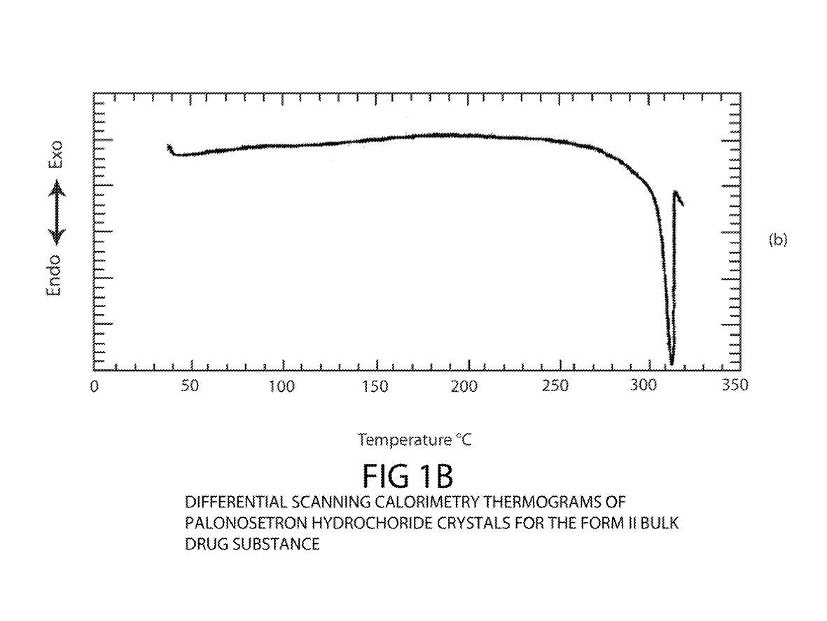

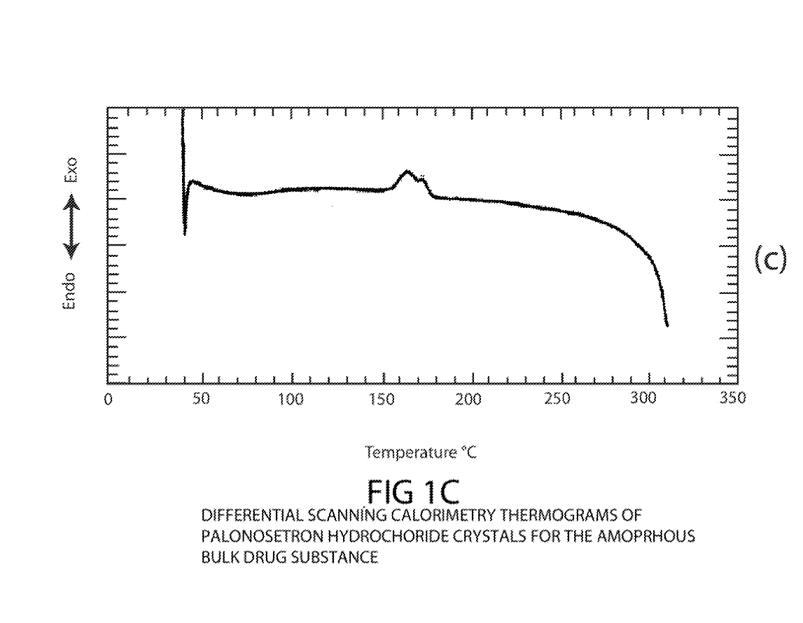

A DSC thermogram of amorphous PH is presented as FIG. 1C, which shows that the glass transition temperature for this phase is in the range of 40° C., and that the material crystallizes in a bimodal progression, with an exotherm onset at ca. 155° C., first exothermic maximum at ca. 163° C., a lesser exothermic maximum at ca. 172° C., and the crystallization essentially complete at about 178° C. The crystalline melting endotherm maximum exceeded 310° C.

Amorphous form PH can also be characterized by the method for obtaining it. Thus, for example, amorphous PH can be characterized as the product obtained by lyophilizing an aqueous solution of PH. Alternatively, as with any amorphous form of an otherwise crystalline substance, amorphous PH can be characterized as the product obtained when melted PH is rapidly quenched to below about 40 or 50° C., thereby bypassing any transition to Forms I or II PH.

### Pharmaceutical Compositions

Various pharmaceutical compositions can be developed that make use of the crystalline and amorphous forms of PH described herein. The composition can be administered by any appropriate route, for example, orally, parenterally, or intravenously, in liquid or solid form. A preferred dose of the compound for nausea or emesis is in the range from about 0.3 to 90 μg/kg of body weight, more preferably from about 0.01 mg. to about 10.0 mg., from about 0.1 mg. to about 2.0 mg., or from about 0.2 mg. to about 1.0 mg. in a single fixed dose (based on the weight of the base). Ideally the active ingredient should be administered to achieve maximum plasma concentrations of the active compound of from about 0.1 to 100 ng/ml, preferably about 1.0 to 50.0 ng/ml, and most preferably about 5-20 ng/ml.

Preferred modes of administrations of the active compound are injectable and oral. These compositions will generally include an inert diluent or an edible carrier. They may be enclosed in gelatin capsules (for oral use) or compressed into tablets (for oral or buccal use) or formulated into troches (for buccal use). For these purposes, the active compound can be incorporated with excipients and used in the form of tablets, troches, or capsules. Pharmaceutically compatible binding agents, and/or adjuvant materials can be included as part of the composition.

Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, orally disintegrating film, orally disintegrating tablet, chewing gum or the like. A syrup may contain, in addition to the active compounds, sucrose as a sweetening agent and certain preservatives, dyes and colorings and flavors.

Solutions or suspensions used for injection can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride, mannitol and dextrose. An injectable preparation can be enclosed in ampoules, disposable syringes or multiple dose vials made of glass or plastic.

### Methods of Treatment

In still further embodiments, the invention provides methods of treating emesis by administering a therapeutically effective amount of one of the crystalline/amorphous forms of PH described herein. The emesis may be acute phase emesis (i.e. emesis experienced within about 24 hours of an emesis inducing event), or delayed emesis (i.e. emesis experienced after the acute phase, but within seven, six, five or four days of an emesis inducing event). The emesis may constitute chemotherapy induced nausea and vomiting (“CINV”) from moderately or highly emetogenic chemotherapy, radiation therapy induced nausea and vomiting (“RINV”), or post-operative nausea and vomiting (“PONV”).

In addition, the morphological forms of PH described herein may be administered in combination (sequentially or simultaneously) with or in alternation with a prophylactic corticosteroid, such as dexamethasone for the purpose of enhancing the anti-nausea and anti-emesis efficacy. They may also be administered in combination (sequentially or simultaneously) with various infusion solutions, including dextrose and saline solutions.

### EXAMPLES

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at room temperature, and pressure is at or near atmospheric. Palonosetron hydrochloride starting material for all tests, unless otherwise specified, can be obtained substantially as described in U.S. Pat. No. 5,510,486.

### Example 1

### Preparation of Polymorphic and Amorphous PH

The Form I polymorph was prepared by crystallization of the compound from an ethanolic solution of palonosetron hydrochloride held at ambient temperature (ca. 25° C.) for one week. The crystals were filtered and dried.

The Form II polymorph was prepared by crystallization of the compound from a hot ethanolic solution of palonosetron hydrochloride. The crystals were filtered immediately upon cooling to room temperature and dried.

The amorphous form was prepared by lyophilization of a solution of the compound in water.

### Example 2

### Methods of Characterizing Crystalline forms

Starting Materials

Polymorphic and amorphous forms of palonosetron hydrochloride for all tests, unless otherwise specified, were obtained substantially as described in the foregoing examples. Palonosetron hydrochloride is nonhygroscopic to humidities up to 82% RH and liquefies at 93% RH after eight days.

Instrumentation

Powder x-ray diffraction patterns were obtained on a Nicolet X-ray diffractometer equipped with a fine focus x-ray tube and a diffracted beam monochromator. The scanning angle was from 3° to 30° 20 at 0.05° per second. All samples were finely ground with a mortar and pestle prior to measurements.

Differential Scanning calorimetry (DSC) thermograms were recorded on a Perkin-Elmer DSC-7 system. The heating rate was 10° C. per minute and the sensitivity range was 5 mcal/second. A nitrogen flow was maintained throughout the runs.

Thermogravimetric analysis (TGA) were conducted on a Perkin-Elmer TGS-2 thermogravimetric balance. The heating was 2.5° C. per minute. A nitrogen purge was maintained throughout each run.

Microscopic observations were made using a Leitz Ortholux II POL-BK polarized light microscope. Thermal Microscopy was conducted on a polarized light microscope in conjunction with a Mettler FP 52 hot stage equipped with a Mettler FP5 control unit. The heating rate used to measure the transition temperature was 10° C. per minute.

Physical Stability Determination—

The physical stability of the polymorphs was determined by wetting the powders with water, suspending in ethanol, ethyl acetate, and exposing them to 93% relative humidity (RH) and 26% RH. The DSC thermogram and the x-ray diffraction pattern were obtained after a few days to determine if phase changes had occurred. The physical stability of the amorphous phase was determined by equilibrating a sample at 40° C., 75% RH for two weeks.

Hygroscopicity Measurement—

Samples of 20-30 mg of the compound were weighed into small weighing bottles with ground glass tops. The bottles were dried in a 60° C. oven for 30 minutes. The samples were cooled in a dessicator containing anhydrous CaSO₄ and then transferred to humidity chambers maintained at constant relative humidities with saturated salt solutions. The samples were weighed at different time intervals until equilibrium was reached.

The percent moisture absorbed at a given time was calculated as follows:

% Weight Change=((Wₜ−Wₒ)/Wₒ)×100

wherein W₁=sample weight at given time after exposure to the indicated relative humidity, and Wₒ=initial sample weight.

Characteristic Power X-Ray Diffraction Pattern Peak Positions

XRDP positions are reported for crystalline forms in terms of the angular positions (two theta) within an allowable variability of plus or minus 0.1°. This allowable variability is specified by the US Pharmacopeia, pages 1843-1844 (1995). The variability of plus or minus 0.1° is intended to be used when comparing two powder X-ray diffraction patterns. In practice, if a diffraction pattern peak from one pattern is assigned a range of angular positions (two theta) which is a measured peak position plus or minus 0.1° and a diffraction pattern peak from the other pattern is assigned a range of angular positions (two theta) which is the measured peak position plus or minus 0.1° and if those ranges of peak positions overlap, then the two peaks are considered to have the same angular position (two theta). For example, if a diffraction pattern peak from one pattern is determined to have a peak position of 5.20°, for comparison purposes the allowable variability allows the peak to be assigned a position in the range of 5.10°-5.30°. If a comparison peak from the other diffraction pattern is determined to have a peak position of 5.35°, for comparison purposes the allowable variability allows the peak to be assigned a position in the range of 5.25°-5.45°. Because there is overlap between the two ranges of peak positions, the two peaks being compared are considered to have the same angular position (two theta).

When assigning characteristic peaks to the polymorphic forms described herein, it will be understood that any one or more of the peaks recited in Tables 1 and 3 can be used to characterize the crystalline form, in any combination, although preferably at least one of the peaks for a crystalline form does not overlap with any of the peaks from an alternative form. In the XRPD patterns depicted in the figures, Cu Kalph2 is eliminated, calculation of d values is performed with wave length 1.5406 Å. Only significant peaks up to 35 °2 theta are listed.

### Example 3

### Characterization of Form I Palonosetron Hydrochloride

Form I consists of bladed or plate-like birefringent crystals. The DSC thermogram shows an endotherm at −313° C. due to melting of the crystals (FIG. 1A). As shown in FIG. 2A and Table 1, Form I displays the following angular positions (two theta) of characteristic peaks in its powder X-ray diffraction pattern: 10.38°±0.1°, 12.04°±0.1°, 14.40°±0.1°, 15.74°±0.1°, 16.89°±0.1°, 17.16°±0.1°, 19.62°±0.1°, 20.88°±0.1°, 23.70°±0.1°, 24.02°±0.1°, 24.73°±0.1° and 25.31°±0.1°.

TABLE 1

| g0396-01.rd | | Palonosetron21000567 | | C |
|---|---|---|---|---|
| Angle | d value | qualtitative | Intensity | Intensity % |
| 2-Theta ° | Angstrom | rel. Intensity | Cps | % |
| 7.05 | 12.5 | m | 513 | 18.7 |
| 10.38 | 8.5 | w | 191 | 7 |
| 12.04 | 7.3 | m | 478 | 17.4 |
| 13.74 | 6.4 | s | 1078 | 39.3 |
| 14.13 | 6.3 | vs | 2741 | 100 |
| 14.40 | 6.1 | m | 593 | 21.6 |
| 15.74 | 5.63 | s | 1717 | 62.6 |
| 16.89 | 5.25 | m | 514 | 18.7 |
| 17.16 | 5.16 | w | 344 | 12.5 |
| 18.40 | 4.82 | s | 1624 | 59.2 |
| 18.68 | 4.75 | w | 260 | 9.5 |
| 19.62 | 4.52 | s | 1005 | 36.7 |
| 19.94 | 4.45 | s | 1517 | 55.3 |
| 20.22 | 4.39 | w | 154 | 5.6 |
| 20.88 | 4.25 | m | 521 | 19 |
| 21.25 | 4.18 | m | 551 | 20.1 |
| 22.34 | 3.98 | vw | 114 | 4.2 |
| 23.22 | 3.83 | s | 1116 | 40.7 |
| 23.70 | 3.75 | s | 843 | 30.8 |
| 24.02 | 3.70 | m | 617 | 22.5 |
| 24.33 | 3.65 | s | 896 | 32.7 |
| 24.73 | 3.60 | w | 395 | 14.4 |
| 25.31 | 3.52 | m | 521 | 19 |
| 26.28 | 3.39 | w | 210 | 7.6 |
| 26.70 | 3.34 | w | 339 | 12.4 |
| 27.67 | 3.22 | m | 772 | 28.2 |
| 29.11 | 3.06 | w | 214 | 7.8 |
| 29.30 | 3.05 | w | 208 | 7.6 |
| 30.13 | 2.96 | m | 583 | 21.3 |
| 31.55 | 2.83 | w | 306 | 11.2 |
| 32.14 | 2.78 | w | 399 | 14.5 |
| 32.78 | 2.73 | w | 180 | 6.6 |
| 33.62 | 2.66 | w | 230 | 8.4 |
| 34.16 | 2.62 | w | 219 | 8 |

For all peak tables:
-Cu Kalpha2 elliminated
-Calculation of d values with wave length 1.5406 A
-signifikant Peaks just up to 35 °2-Theta listed

Form I was stable as a suspension in water, ethanol or ethyl acetate. This form deliquesced after two weeks at 93% relative humidity at ambient temperature (˜25° C.).

The results of the hygroscopicity study are summarized in Table 2. Form I was not hygroscopic at humidities up to 82%, absorbing 0.5% water after eight days. At 93% RH, the drug absorbed 26% moisture after eight days turning into a paste. The drug liquefied after being held under these conditions for two weeks.

TABLE 2
Water Absorption of Form I

| Relative | Time | Water absorbed |
|---|---|---|
| Humidity (%) | (days) | (weight %) |
| 50 | 1 | 0.02 |
| | 4 | 0.14 |
| | 8 | 0.05 |
| 75 | 1 | 0.15 |
| | 4 | 0.22 |
| | 8 | 0.22 |
| 82 | 1 | 0.14 |
| | 4 | 0.25 |
| | 8 | 0.54 |
| 93 | 1 | 0.07 |
| | 4 | 0.63 |
| | 8 | 25.7 |
| | | Semi |
| | | liquid |
| | 15 | Liquid |

### Example 4

### Characterization of Form II Palonosetron Hydrochloride

The Form II polymorph exhibits properties similar to Form I crystals. The DSC thermogram shows only a melting endotherm at ca. 311.7° C. (FIG. 1B). The powder X-ray diffraction pattern of Phase II is shown in FIG. 2B and Table 3. As shown in FIG. 2B and Table 3, Form II displays the following angular positions (two theta) of characteristic peaks in its powder X-ray diffraction pattern: 9.92°±0.1°, 11.35°±0.1°, 12.98°±0.1°, 15.38°±0.1°, 16.14°±0.1°, 17.51°±0.1°, 25.08°±0.1°, 28.89°±0.1°, 29.66°±0.1° and 32.39°±0.1°.

TABLE 3

| g0982-01.rd | | Palonosetron 08/27/2 | | |
|---|---|---|---|---|
| Angle | d value | qualtitative | Intensity | Intensity |
| 2-Theta ° | Angstrom | rel. Intensity | Cps | % |
| 7.05 | 12.5 | vw | 98.9 | 4.1 |
| 9.92 | 8.9 | w | 206 | 8.6 |
| 11.35 | 7.8 | w | 150 | 6.2 |
| 12.98 | 6.8 | s | 746 | 31 |
| 13.69 | 6.5 | m | 468 | 19.5 |
| 14.15 | 6.3 | m | 571 | 23.8 |
| 15.38 | 5.76 | s | 754 | 31.3 |
| 16.14 | 5.49 | m | 438 | 18.2 |
| 17.51 | 5.06 | m | 569 | 23.7 |
| 18.41 | 4.82 | s | 1185 | 49.3 |
| 19.93 | 4.45 | vs | 2404 | 100 |
| 21.31 | 4.17 | vw | 93.9 | 3.9 |
| 22.11 | 4.02 | w | 273 | 11.4 |
| 23.18 | 3.83 | s | 935 | 38.9 |
| 23.49 | 3.79 | w | 268 | 11.1 |
| 24.38 | 3.65 | m | 601 | 25 |
| 25.08 | 3.55 | s | 748 | 31.1 |
| 26.17 | 3.40 | w | 152 | 6.3 |
| 27.55 | 3.23 | m | 587 | 24.4 |
| 28.89 | 3.09 | w | 249 | 10.3 |
| 29.66 | 3.01 | w | 193 | 8 |
| 30.03 | 2.97 | vw | 101 | 4.2 |
| 31.50 | 2.84 | w | 332 | 13.8 |
| 31.96 | 2.80 | w | 240 | 10 |
| 32.39 | 2.76 | m | 391 | 16.3 |
| 33.69 | 2.66 | w | 197 | 8.2 |
| 34.13 | 2.62 | w | 183 | 7.6 |
| 34.53 | 2.60 | w | 165 | 6.9 |

Form II crystals were converted to Form I when suspended in ethanol for one week at ambient temperature (˜25° C.).

### Example 5

### Characterization of Amorphous Palonosetron Hydrochloride

The DSC thermogram shows that the glass transition temperature for this phase is in the range of ca. 40° C., and that the material crystallizes in a bimodal progression, with an exotherm onset at ca. 155° C., first exothermic maximum at ca. 163° C., a lesser exothermic maximum at 172° C. exotherm peak, and the crystallization was essentially complete at about 178° C. under the conditions of the thermal analysis; the melting endotherm of the crystals thus formed peaks above 310° C. (FIG. 1C). The X-ray powder diffraction pattern of the unheated product of lyophilization is characteristic of an amorphous material (FIG. 2C), that is, the XRD peak maxima are diffuse and of low intensity; here the characteristic angular positions (two theta) are at 16°±0.3° and 21°±0.3°.

The amorphous form remained amorphous at 26% RH after two weeks. However, this material deliquesced immediately after it was removed from the desiccator and exposed to ambient humidity, and it subsequently re-solidified as Form II. By contrast, at 40° C., 75% RH for two weeks the amorphous form was converted to Form I.

### Example 6

### Representative Gelcap Formulation

Table 4 describes representative formulations for a gelcap solid oral dosage form containing 0.25, 0.50 and 0.75 mg. of palonosetron.

TABLE 4
Representative Gelcap Formulation

| | Formula (mg per capsule) | | |
|---|---|---|---|
| Names of Ingredients | 0.25 mg | 0.50 mg | 0.75 mg |
| Active drug substance | 0.28^{a} | 0.56^{b} | 0.84^{c} |
| Palonosetron HCl | | | |
| Excipients | 5.57 | 5.57 | 5.57 |
| Purified water | | | |
| Glycerin, anhydrous | 6.40 | 6.40 | 6.40 |
| Butylated hydroxyanisole (BHA) | 0.13 | 0.13 | 0.13 |
| Polyglyceryl oleate (Plurol Oleique | 6.65 | 6.65 | 6.65 |
| CC 497) | | | |
| Mono- and di-glycerides of | 113.97 | 113.69 | 113.41 |
| Capryl/Capric Acid (Capmul MCM) | | | |
| Nitrogen | — | — | — |
| Theoretical fill weight | 133.00 mg | 133.00 mg | 133.00 mg |
| Gelatin Capsule Shell, #3, oval | 1 capsule | 1 capsule | 1 capsule |
| (Cardinal Health)^{d} | | | |

^{a}corresponding to 0.25 mg free base
^{b}corresponding to 0.50 mg free base
^{c}corresponding to 0.75 mg free base
*The amounts of the two excipients reported in parentheses are those used for the phase 3 formulations

### Example 7

### Representative Injectable Formulation

The following Table 5 describes a representative injectable formulation containing palonosetron.

TABLE 5
Representative Injectable Formulation

| Ingredient | mg/mL |
|---|---|
| Palonosetron Hydrochloride | 0.05 |
| Mannitol | 41.5 |
| EDTA | 0.5 |
| Trisodium citrate | 3.7 |
| Citric acid | 1.56 |
| WFJ | 1.0 |
| Sodium hydroxide solution and/or | pH 5.0 ± 0.5 |
| hydrochloric acid solution | |
| Flavoring | q.s. |

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-35. (canceled)

36) Amorphous palonosetron hydrochloride.

37-38. (canceled)
